(19) United States
(12) Patent Application Publication
(10) Pub. No.: US 2026/0137902 A1
(43) Pub. Date: May 21, 2026

(54) INTRATUMORAL INFUSION CATHETER

(71) Applicant: City of Hope, Duarte, CA (US)

(72) Inventor: Franz E. Boas, Duarte, CA (US)

(21) Appl. No.: 19/393,213

(22) Filed: Nov. 18, 2025

Related U.S. Application Data
(60) Provisional application No. 63/723,381, filed on Nov. 21, 2024.

(51) Int. Cl. A61M 25/00; A61M 25/04; A61M 39/24

(52) U.S. Cl. CPC A61M 25/0075; A61M 25/0009; A61M 25/0054; A61M 25/0068; A61M 25/04; A61M 39/24; A61M 2025/006; A61M 2025/0076; A61M 2202/0007; A61M 2202/049; A61M 2205/0216; A61M 2205/04; A61M 2205/32; A61M 2205/3334; A61M 2210/02; A61M 2210/1039; A61M 2210/1071; A61M 2210/1082; A61M 2210/166

(57) ABSTRACT

An implantable intratumoral infusion catheter comprises an intratumoral catheter formed of an elongated body having an internal lumen. An anchor mechanism is coupled to the elongated body. The anchor mechanism is configured to interact with a tumor or surrounding tissue to retain the catheter in the tumor.

### CROSS REFERENCE TO RELATED APPLICATION

The present application claims priority to U.S. Provisional Application No. 63/723,381, filed on Nov. 21, 2024, and titled “Intratumoral Infusion Catheter,” the entirety of which is incorporated by reference herein.

### BACKGROUND

Intratumoral injection of immunotherapy agents can be used to generate in situ cancer vaccines, resulting in abscopal effects, with regression of uninjected tumors. Local delivery of immunotherapy agents can result in local drug concentrations that are hundreds to thousands of times higher than the systemic drug concentration. Local delivery thus enables use of drugs that are too toxic or are ineffective when delivered systemically, such as vaccine adjuvants or cytokines.

However, daily intratumoral injections and complex dosing schedules involving different drugs at different time points may be required to achieve the best results. In an example scenario such as a highly perfused liver tumor, the injected drug rapidly washes out into the systemic circulation. Thus, repeated injections can be required to generate sufficient immune stimulation.

### SUMMARY

Timed local delivery of different drugs during different phases of a cancer-immunity cycle, from antigen presentation to effector cell recruitment and activation, can improve the anti-tumor immune response. Although the optimal timing of drug delivery is still unclear, some data suggests that immunosuppressive signals should be blocked before immunogenic cell death, T cell costimulation should occur after immunogenic cell death, dendritic cells should be recruited to the tumor before they are activated, and antigen presentation should be promoted before stimulating T cells.

Repeated intratumoral injections have been performed in early phase human trials but are not practical for widespread use. To address this issue, disclosed is an intratumoral infusion catheter that can be connected to one or more subcutaneous ports. The catheter can be implanted in locations such as tumors in the liver and other organs. For example, the catheter can be implanted in a liver, lung, kidney, pancreas, lymph node, prostate, bone, muscle, or fat. This allows for long-term intratumoral infusion of multiple drugs, using arbitrary dosing schedules, such as via a subcutaneous port currently used for intravenous infusions. In an implementation, at least a portion of the catheter is tunneled through subcutaneously.

In one aspect, there is disclosed an implantable intratumoral infusion catheter, comprising an intratumoral catheter formed of an elongated body having an internal lumen; and an anchor mechanism coupled to the elongated body, the anchor mechanism configured to interact with a tumor or surrounding tissue to retain the catheter in the tumor.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

These and other features will be more readily understood from the following detailed description taken in conjunction with the accompanying drawings, in which:

FIGS. 1 and 2 show representations of an intratumoral catheter system configured for tumor immune modulation via infusion of an infusate into or near a tumor location of a patient.

FIGS. 3 and 4 show schematic representations of a region the catheter positioned in a tumor.

FIG. 5 shows a representation of a distal portion of a catheter.

FIG. 6 shows a cutaway view of a portion of the catheter.

FIG. 7 shows several implementations of the catheter with variations in diameter of at least one side hole.

FIG. 8 shows several implementations of the catheter with variations in position or location of the side hole.

FIG. 9 shows several implementations of the catheter with variations in an angle of a side hole/barb.

FIG. 10 shows several implementations of the catheter with variations in barb spiral angle.

FIGS. 11-14 show various implementations of retention structures for a catheter.

FIGS. 15-17 show various implementations of side holes for a catheter.

FIGS. 18-19 show various implementations of end hole occlusion for a catheter.

FIGS. 20-21 show various implementations of barb geometry for a catheter.

FIGS. 22-24 show various implementations of suture attachment configurations for a catheter.

FIG. 25 shows a catheter having a removable stiffener.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Disclosed are implementations of an intratumoral catheter and associated methods of use. The catheter is configured to infuse an infusate, such as a medication, to a target anatomical location such as, for example, in a tumor of a patient. The catheter includes one or more retention structures configured to retain the catheter in a fixed or substantially fixed position relative to target anatomy including tissue. In example implementations, the retention structures are barbs that extend outward from a catheter body and that can vary in size and geometry. The catheter also includes an internal lumen and one or more side holes for infusing the infusate from the catheter to or towards the target anatomy. The catheter can include a distal end opening that communicates with the internal lumen. The distal end opening can be selectively occluded. A suture and/or a stiffening member, such as a stylet, can be removably or fixedly positioned in the internal lumen of the catheter. In an implementation, infusate is delivered from the catheter to the target anatomy in a pulsatile flow manner. The catheter can be coupled to an infusion pump. The catheter can be used to infuse any of a variety of therapies including, for example, cytotoxic chemotherapy, targeted therapy, antibody, radioisotope, chemical ablation agent, immunotherapy agents (such as checkpoint inhibitors, vaccine adjuvants, immune stimulants, viruses, polymers), cellular therapies, exosomes, or other drugs.

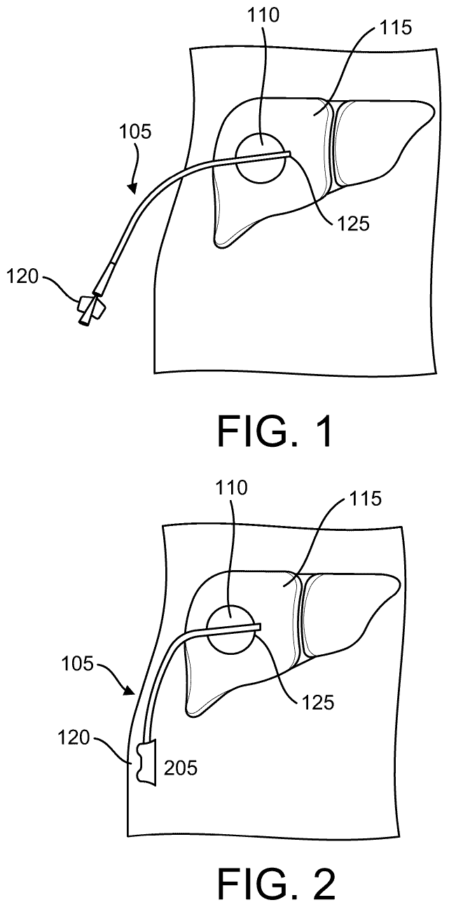

FIG. 1 shows a representation of an intratumoral catheter system configured for tumor immune modulation via infusion of an infusate into or near a tumor location of a patient. The system is described in an example context of treating a tumor in a liver. However, it should be appreciated that the system can be used for infusion and/or treatment to other locations of a patient as well as for non-tumor infusion. The catheter system is configured to be entirely or partially implanted within a patient's body and retained at a target location in a patient. The catheter system is configured to remain in or near target tissue even with breathing motion of the patient. In an implementation, at least a portion of the catheter is tunneled through subcutaneously.

With reference still to FIG. 1, which is not necessarily to scale, a catheter 105 is formed of an elongated body, such as a tubular body, having an internal lumen through which infusate can flow to a tumor 110, such as a tumor in a liver 115. The catheter 105 has a proximal region or proximal end 120 and a distal end 125. In the implementation of FIG. 1, the proximal end 110 has an extracorporeal connector, such as a Luer connector, that can be removably and fluidly coupled to a source of infusate that passes through the catheter 105 to the tumor 115. In another implementation shown in FIG. 2, a subcutaneous port 205 is located at the proximal end 120. The subcutaneous port 205 resides within a body of the patient and provides or is coupled to a source of infusate for the catheter 105. The subcutaneous port 205 can be accessed with a Huber needle for example.

The catheter 105 has one or more side holes along the length of its elongated body through which fluid can flow out of the catheter 105 (from the internal lumen) into or toward the tumor 110. The configuration of the side holes can vary with some example implementations described herein. The side holes can extend at least partially through adjacent retention structures such as through barbs. Or the side holes can extend through a side wall of the catheter 105. In another implementation, the side holes can be positioned at least partially through a longitudinal cylinder underneath the barbs.

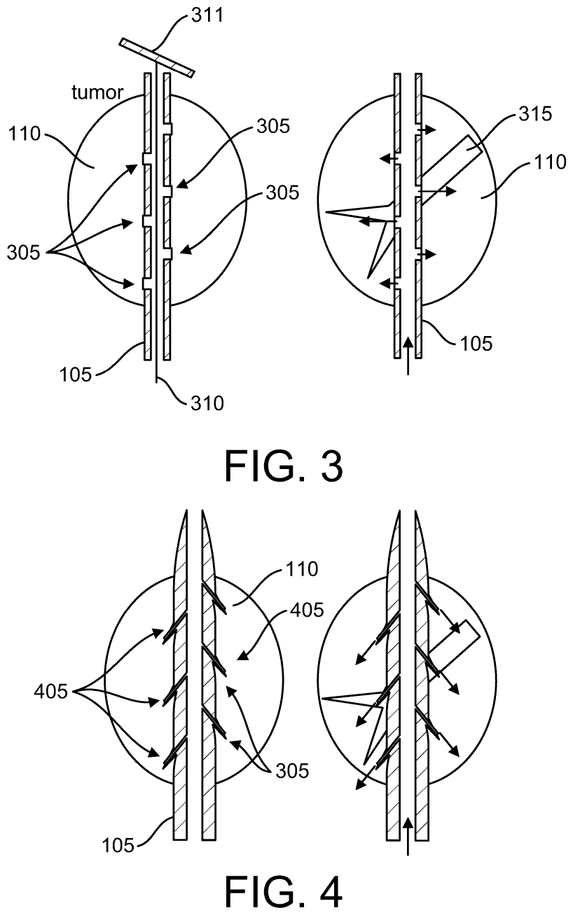

FIG. 3 shows a schematic representation of a distal region of the catheter 105 positioned in a tumor 110. The catheter has one or more side holes 305 that communicate with the tumor 110. At least one suture 310 optionally extends through the internal lumen of the catheter 105. A distal end of the suture 310 connects to an anchor 311 positioned just outside a distal end of the catheter 105. The anchor 311 interacts with anatomy to anchor, fixe, or otherwise secure a position of the catheter 105 relative to target anatomy such as the tumor 110. The suture 310 and anchor 311 retain the catheter 105 in position such as in position relative to a liver. As shown in the right-side diagram of FIG. 3, infused fluid flows into tumor 110 from the catheter 105 via the side holes 305. The anchor can be made of various materials including metal (such as for example steel, nickel-titanium alloy) or polymers. The suture and the anchor can be attached to the port. As described further below, the side holes can vary in size, shape, location, and quantity.

FIG. 4 shows an implementation of the catheter 105 that has one or more retention structures such as barbs 405 (or prongs, protrusions, thorns, etc) positioned along the length of the catheter 105. At least one of the barbs 405 is positioned at or near a corresponding side hole 305. The barbs 405 anchor the catheter 105 to the tumor 110 or to other anatomy. The side holes 305 are configured to regulate the flow rate of fluid therethrough to provide regulated fluid flow, such as uniform fluid flow, to achieve drug delivery into the tumor 110. The side holes 305 can vary in configuration. For example, one or more side holes 305 can be positioned immediately adjacent a corresponding barb 405. Or a side hole 305 can extend entirely or partially through a barb 305 itself to form a tunnel through the barb that provides fluid communication between the catheter's internal lumen to a location external to the catheter 105. The barbs 405 retain the catheter in the tumor and the side holes enable intratumoral drug infusion. As described further below, the barbs or other catheter anchor mechanisms can vary in size, shape, location, and quantity.

Intratumoral drug delivery can be improved by increasing the fluid flow resistance through the side hole (for example, such as by using smaller side holes) or by increasing the flow rate (for example, using pulsatile flow). This results in higher pressure in the catheter, which overcomes the tissue pressure, resulting in more drug delivery into tumor.

In a non-limiting example, a force required to pull the catheter out of the tissue, such as liver tissue, is 7.6 N for the sidehole catheter with suture anchor and 5.3 N for the barbed sidehole catheter.

The catheter 105 can be manufactured pursuant to various manufacturing processes. In an example implementation, the catheter 105 is manufactured pursuant to a three-dimensional printing process such as by using microstereolithography.

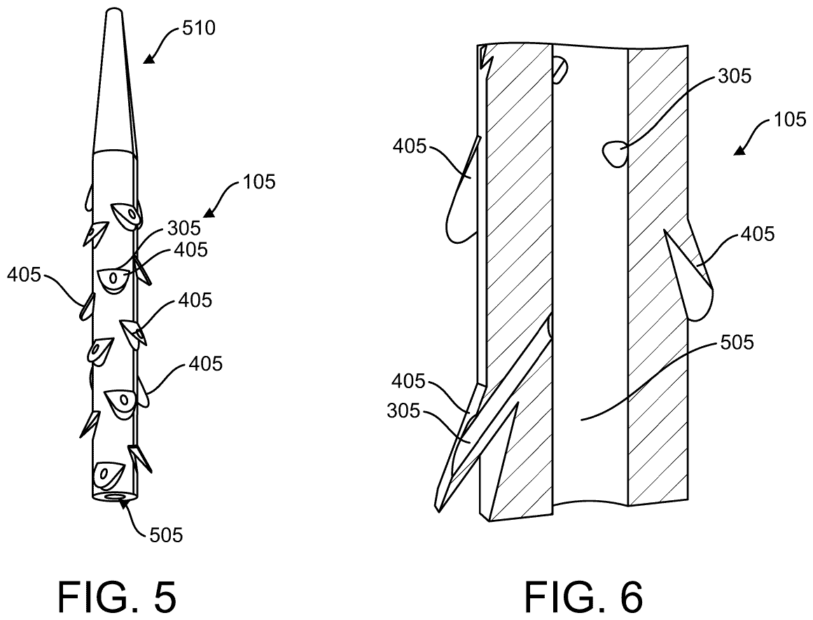

FIG. 5 shows a representation of a distal portion of a catheter 105. The internal lumen 505 extends through the catheter 105. The catheter 105 has a tapered, distal end 510 with an optional distal opening (such as at or near the catheter's distal-most end) that communicates with the internal lumen 505. As discussed, the barbs 405 are distributed along the length of at least a portion of the catheter 105. A corresponding opening 305 is positioned at or near a respective barb 405. FIG. 6 shows an example, cutaway view of a portion of the catheter 105 showing the lumen 505, which extends through and along the length of the catheter 105. One or more of the barbs 405 has an associated passageway or opening 305 that extends along, through, or near the respective barb 405. The opening 305 has one end that communicates with the internal lumen 505 and another end that communicates to a location external to the catheter 105. The opening 305 can form or be part of a lumen of tunnel that extends through the barb 405.

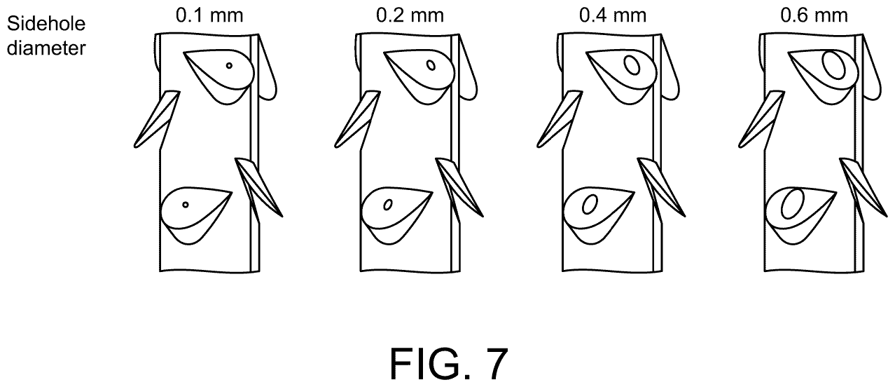

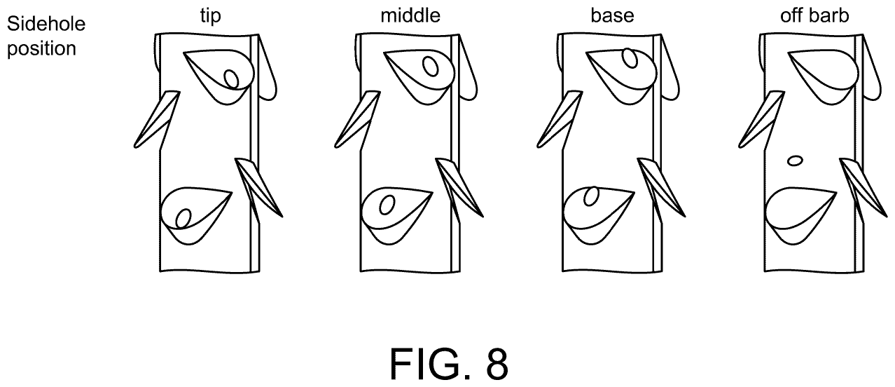

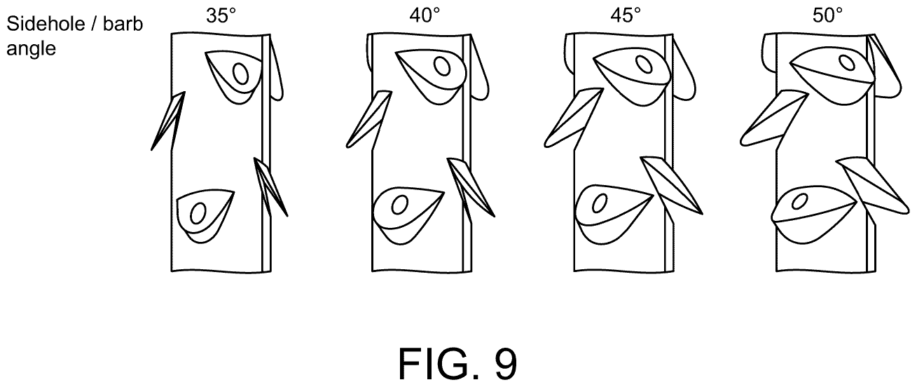

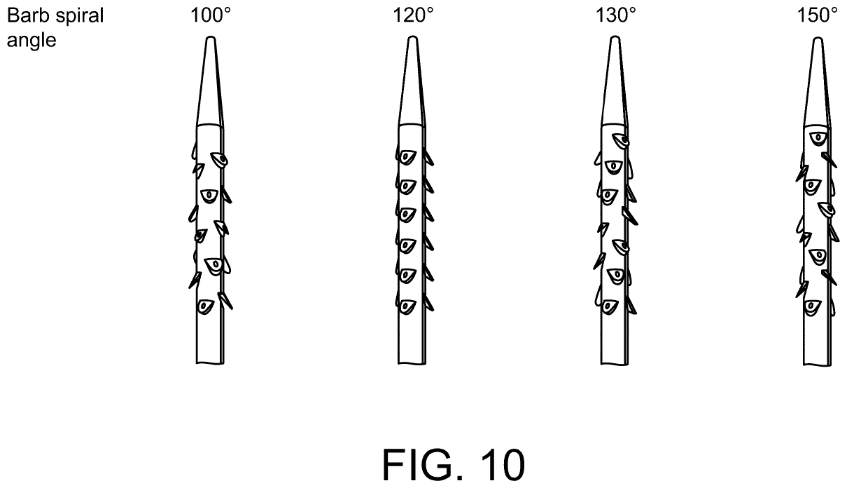

The configuration of the barbs 405 and side holes 305 can vary. Some example aspects that can vary include for example, side hole diameter, side hole position relative to the barb, and an angle of the barb relative to a long axis of the catheter. FIG. 7 shows several implementations of the catheter with variations in diameter of the side hole and shows non-limiting example of a diameter of the side holes. FIG. 8 shows several implementations of the catheter with variations in position or location of the side hole. For example, the side holes can be positioned at various locations of the barbs or can be positioned adjacent the barbs. FIG. 9 shows several implementations of the catheter with variations in an orientation angle of the barbs relative to a long axis of the catheter. That is, a barb can be defined by a long axis that extends at a predetermined angle relative to the long axis of the catheter. FIG. 10 shows several implementations of the catheter 105 with variations in barb spiral angle. Other aspects can vary as well.

When infusing drug through a multi-side hole catheter into a tumor, one or more of the side holes can be in low resistance blood vessels, and one or more side holes can be embedded in tumor tissue. When the flow rate is low, or when side holes are large, all the fluid can possibly go down the path of least resistance, which is into the veins, and not into the tumor. Intratumoral drug delivery can be improved by increasing the side hole resistance (for example, smaller side holes), or by increasing the flow rate (for example, pulsatile flow). This results in higher pressure in the catheter, which overcomes the tissue pressure, resulting in more drug delivery into tumor.

In an implementation, the catheter has a cuff (such as a fabric cuff), textured surface, or adhesive surface that can be used to retain the catheter in the tumor. For example, a cuff can extend outwardly from the catheter such that the cuff interacts with tissue to retain the catheter in place. Or the surface of the catheter can be textured such that friction retains the catheter in place. The catheter may also have adhesive on an outer surface to retain the catheter in place. In an implementation, the retention mechanism for a tunneled dialysis catheter is a small piece of fabric that is glued to the catheter. Tissue attaches to this rough fabric surface, and increases the force required to pull out the catheter, although the catheter is still removable from the body upon application of a sufficiently strong pulling force on the catheter. In an implementation, the fabric cuff comes out of the body with the catheter. The fabric cuff may be disengaged from the catheter and retained in the patient when the catheter is removed in an implementation. In an implantation, the fabric cuff has a structure such as the loop portion of a hook-and-loop fastener, such as VELCRO.

### Example Retention Mechanisms

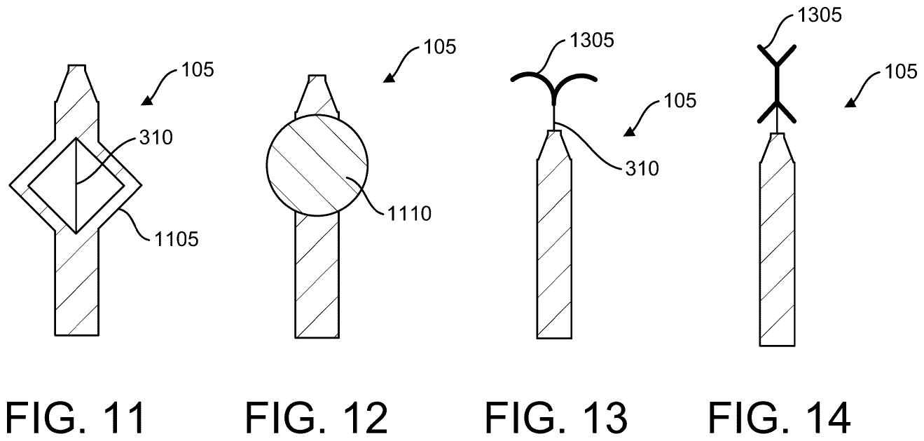

As discussed, the catheter can have one or more retention mechanisms such as barbs. Other implementations of catheter retention structures or mechanisms are now described. FIG. 11 shows a schematic representation of the catheter 105 wherein the catheter 105 is a Malecot catheter. The catheter 105 has a suture 310 that extends through the catheter. The suture 310 can be pulled, tensioned, or manipulated to cause an anchor portion 1105 of the catheter 105 to expand outward and form an anchor. FIG. 11 shows the anchor in an expanded state such that the anchor has a widened width relative to a width of the remainder of the catheter. In a non-expanded state, the anchor can have a width that is flush with a width of the remainder of the catheter.

FIG. 12 shows the catheter 105 having an expandable element, such as a balloon 1110, that can expand outward (in an expanded state) to form an anchor relative to surrounding tissue.

FIGS. 13 and 14 shows the catheter 105 having a suture 310 that extends through the catheter 105. The suture 105 connects to a distal anchor 1305. The anchor 1305 is pushed through the catheter in a collapsed configuration. After being deployed in tissue beyond the distal tip of the catheter, the anchor 1305 expands into an expanded, deployed configuration.

### Example Side Hole Configurations

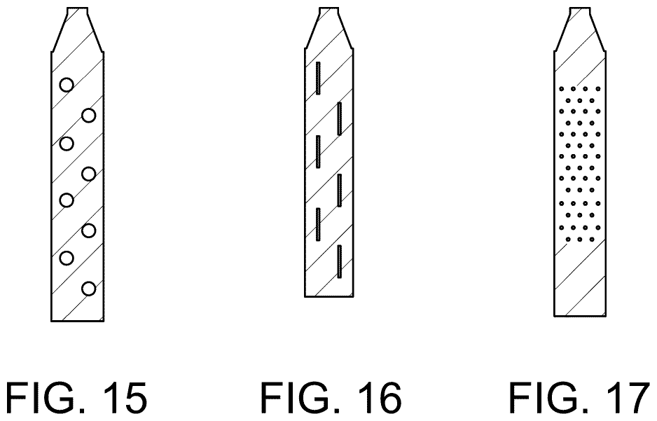

The side holes can also vary in configuration, such as in size, shape, and spacing, and location. FIG. 15 shows an implementation of the catheter wherein the side holes are circular in cross section. FIG. 16 shows an implementation of the catheter wherein the side holes are shaped as elongated slits. FIG. 17 shows an implementation of the catheter wherein the side holes are micro holes such as having a diameter or less than 0.1 mm. The size and shape of the side holes can vary in a single catheter.

### Example End Hole Configurations

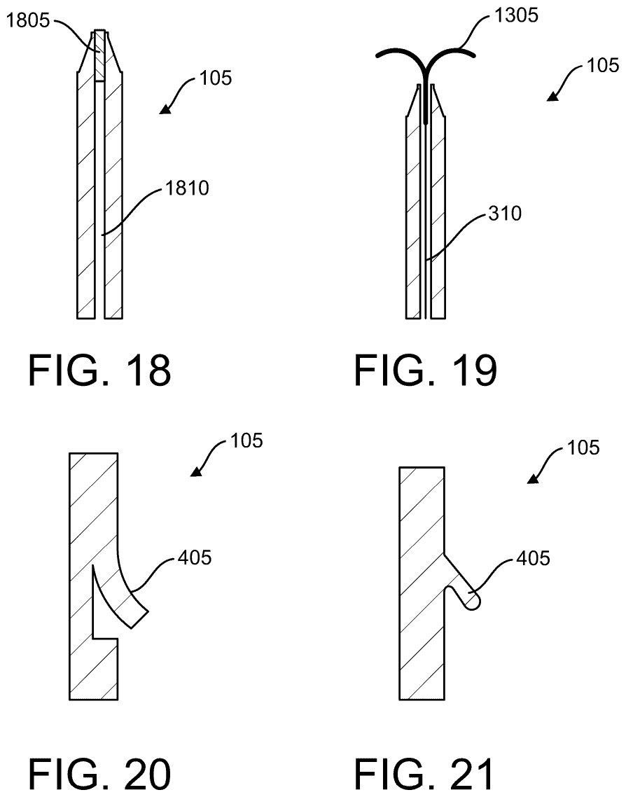

As mentioned, the catheter 105 can optionally have a distal opening or hole located at a distal end of the catheter 105. FIG. 18 shows an implementation of the catheter 105 having an internal lumen 1810 that communicates with a distal end hole. An occlusion element 1805 such as a pin or dowel or wire can be removably positioned in the end hole. The occlusion element 1805 can optionally be connected to a suture or wire 310, which can also be optionally connected to an anchor 1305 as shown in FIG. 19. The end hole can also be valved. That is, a valve can be positioned within the end hole such that the valve regulates a fluid flow rate through the end hole. The valve can be opened and closed to permit or block flow through the end hole. In an implementation, the valve is a one-way valve.

### Example Barb Geometry

In an implementation where the catheter 105 has one or more barbs, the configuration of the barbs can vary. FIG. 20 shows an implementation wherein at least one barb 405 is formed by a flap that has been cut out of a tubular portion of the catheter 105. In such an embodiment, the catheter 105 is initially a tube, and a side wall of the catheter is cut to form a flap and form an opening that communicates with the catheter's internal lumen. FIG. 21 shows an implementation wherein the barb 405 is formed of a fin structure that extends outward from a side wall of the catheter 105. The fin barb can be formed such as via a molding process.

### Example Suture Attachment Configurations

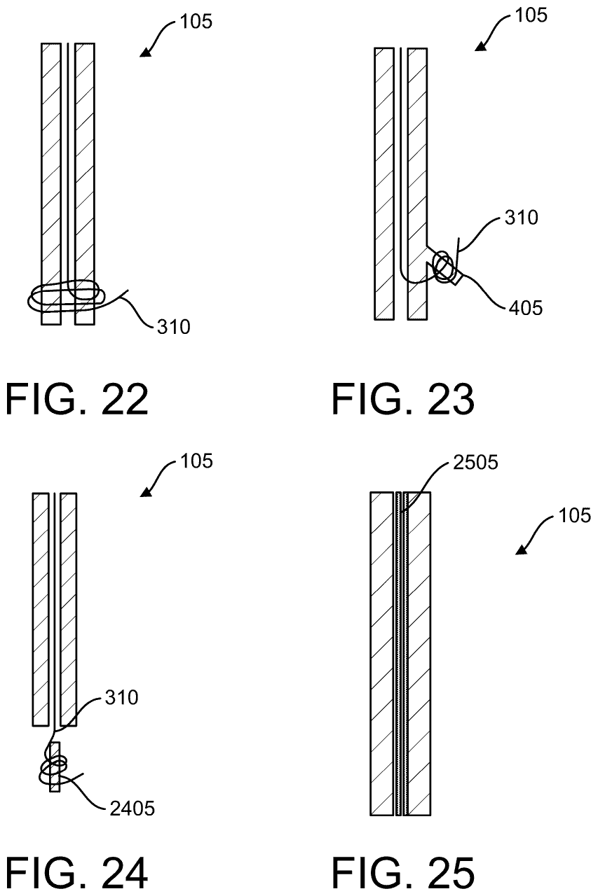

The suture 310 can be attached to the catheter 110 in various manners. FIG. 22 shows the suture 310 extending through the catheter 105. An end of the suture 310 is wrapped around the proximal end of the catheter 105. In an implementation shown in FIG. 23, the suture 310 wraps around post 405 that extends outward at the proximal end of the catheter 105. In an implementation shown in FIG. 24, the suture 310 is attached to a connector 2405 for the port reservoir, then the port reservoir is connected to the catheter.

FIG. 25 shows an implementation wherein a stiffening member 2505, such as a stylet, is positioned within the catheter's inner lumen during catheter placement, and the stiffening element is removed after placement. The stiffening member has a stiffness that is greater than a stiffness of the catheter.

In use, the catheter is positioned in a body of the patient such that a distal region of the catheter is positioned in or near the tumor. A proximal end of the catheter may be positioned outside the patient's body, as shown in FIG. 1. Or the proximal end may be positioned inside the body, as shown in FIG. 2. Fluid is infused through the internal lumen of the catheter and the fluid flow out of the internal lumen via the one or more side holes 305.

Certain exemplary implementations have been described to provide an overall understanding of the principles of the structure, function, manufacture, and use of the systems, devices, and methods disclosed herein. One or more examples of these implementations have been illustrated in the accompanying drawings. Those skilled in the art will understand that the systems, devices, and methods specifically described herein and illustrated in the accompanying drawings are non-limiting exemplary implementations and that the scope of the present invention is defined solely by the claims. The features illustrated or described in connection with one exemplary implementation may be combined with the features of other implementations. Such modifications and variations are intended to be included within the scope of the present invention. Further, in the present disclosure, like-named components of the implementations generally have similar features, and thus within a particular implementation each feature of each like-named component is not necessarily fully elaborated upon.

One skilled in the art will appreciate further features and advantages of the disclosure based on the above-described implementations. Accordingly, the present application is not to be limited by what has been particularly shown and described, except as indicated by the appended claims. Any publications and references cited herein are expressly incorporated by reference in their entirety.

1. An implantable intratumoral infusion catheter, comprising:
an intratumoral catheter formed of an elongated body having an internal lumen; and
an anchor mechanism coupled to the elongated body, the anchor mechanism configured to interact with a tumor or surrounding tissue to retain the catheter in the tumor.

2. The catheter of claim 1, wherein the intratumoral catheter is connected to a subcutaneous port reservoir.

3. The catheter of claim 1, wherein the intratumoral catheter is radiopaque and contains at least one side hole or slit.

4. The catheter of claim 3, wherein the side holes are 0.5 mm in diameter or less.

5. The catheter of claim 3, wherein the side holes are 0.1 mm in diameter or less.

6. The catheter of claim 3, wherein the catheter has a distal tip that is 3D printed.

7. The catheter of claim 3, further comprising a cylindrical pin or wire that occludes an end hole of the catheter.

8. The catheter of claim 3, wherein the end hole is valved.

9. The catheter of claim 1, wherein the anchor mechanism comprises at least one barb or ridge on the catheter, wherein the barb or ridge is configured to retain the catheter in a tumor.

10. The catheter of claim 9, wherein a side hole extends through at least one barb or ridge.

11. The catheter of claim 1, wherein a suture anchor is used to retain the catheter in the tumor.

12. The catheter of claim 11, wherein the anchor mechanism further comprises a suture anchor that extends through the internal lumen and occludes the end hole.

13. The catheter of claim 1, wherein the anchor mechanism comprises a balloon or Malecot mechanism.

14. The catheter of claim 1, further comprising an inner stiffener that extends through the internal lumen and that is configured to assist in advancement of the catheter into the tumor.

15. The catheter of claim 2, wherein the distal end of the catheter that is advanced into the tumor is stiff, and the proximal end of the catheter that is attached to the port is more elastic.

16. The catheter of claim 1, wherein the catheter is placed in a tumor in the liver, lung, kidney, pancreas, lymph node, prostate, bone, muscle, or fat.

17. The catheter of claim 1, wherein cytotoxic chemotherapy, targeted therapy, antibody, radioisotope, chemical ablation agent, immunotherapy agents (such as checkpoint inhibitors, vaccine adjuvants, immune stimulants, viruses, polymers), cellular therapies, exosomes, or other drugs are infused through the catheter.

18. The catheter of claim 1, wherein an infusion pump is used to infuse drug into the catheter.

19. The catheter of claim 18, wherein the infusion pump is programmed to infuse drug at a rate that varies over time.

20. The catheter of claim 19, wherein the drug is infused in a pulsatile fashion.

21. The method of claim 1, wherein a portion of the catheter is tunneled subcutaneously through tissue.

22. The method of claim 1, wherein a fabric cuff, textured surface, or adhesive surface is used to retain the catheter in the tumor.
